(19) United States
(12) Patent Application Publication
(10) Pub. No.: US 2019/0282416 A1
(43) Pub. Date: Sep. 19, 2019

(54) DIAPER WITH AN ENCLOSED ACCESSORY

(71) Applicant: Christine Adams, Putnam, CT (US)

(72) Inventor: Christine Adams, Putnam, CT (US)

(21) Appl. No.: 16/152,724

(22) Filed: Oct. 5, 2018

Related U.S. Application Data
(60) Provisional application No. 62/645,055, filed on Mar. 19, 2018.

(51) Int. Cl. A61F 13/84

(52) U.S. Cl. CPC A61F 13/84; A61F 2013/8476; A61F 2013/8497

(57) ABSTRACT

A diaper with an enclosed accessory having an internal absorbent layer and an external impermeable layer. The diaper includes an accessory compartment strip attached to the external impermeable layer. The accessory compartment strip includes a pull tab external layer attached to an internal moisture retaining enclosure which retains an accessory. The moisture retaining enclosure includes a bottom layer attached directly to the external impermeable layer and a top release layer attached directly to the pull tab layer, wherein the top release layer releases the enclosed accessory when the pull tab is removed.

### CROSS-REFERENCE TO RELATED APPLICATION

This application claims the benefit of priority of U.S. Provisional Application No. 62/645,055 filed on Mar. 19, 2018, the disclosure of which is incorporated herein by reference in its entirety.

### FIELD OF THE INVENTION

Embodiments described herein generally relate to diapers, and more particularly to a diaper with an enclosed accessory.

### BACKGROUND OF THE INVENTION

A diaper is an undergarment that is worn in most cases by an infant or baby that absorbs any waste in terms of urine or defecation. The diaper is designed to absorb the waste product and to avoid soiling of the outside clothing or exterior area. Diapers need to be regularly changed after soiling. Although diapers are primarily used by infants some adults with bladder control problems may also wear a diaper to avoid soiling of clothing. Most diapers used today are disposable-type diapers that are used once and then thrown away. The outer portion is usually a plastic and the inner portion is an absorbent material.

The duty of diaper changing can be complicated, messy, and even a stressful experience, but a necessary one, nonetheless. Moreover, diaper changing requires multiple materials which can be a hassle to manage when the consumer does not have time to organize their supplies or is in a public place. Many times, caregivers use wet wipes as a means to wipe the area covered by the diaper to prevent an infection. A wet wipe is a small wiping cloth usually of manmade materials that includes moisture to wipe the area and is usually stored in a separate container. Thus, it is desirable to facilitate a more convenient diaper changing experience, by combining the diaper and the wipe together in one composite unit.

### BRIEF DESCRIPTION OF THE DRAWINGS

The various advantages of the embodiments of the present disclosure will become apparent to one skilled in the art by reading the following specification and appended claims, and by referencing the following drawings, in which:

FIG. 1 shows an exemplary diaper with an enclosed accessory according to an embodiment of the present disclosure.

FIG. 2 shows an exemplary view of an enclosed accessory removed from the diaper with an enclosed accessory shown in a folded position according to an embodiment of the present disclosure.

FIG. 3 shows a cross sectional view of a diaper with an enclosed accessory according to an embodiment of the present disclosure.

### SUMMARY OF THE INVENTION

Exemplary embodiments disclosed herein describe a diaper with an enclosed accessory. The diaper has an internal absorbent layer and an external impermeable layer having an accessory compartment strip attached thereto. The accessory compartment strip includes a pull tab external layer attached to an internal moisture retaining enclosure which retains an accessory. The moisture retaining enclosure has a bottom layer attached directly to the external impermeable layer and a top release layer attached directly to the pull tab layer. The top release layer releases the enclosed accessory when the pull tab is removed.

In some exemplary embodiments, the accessory compartment strip is located on a front side of the external impermeable layer of the diaper.

In some exemplary embodiments, the accessory compartment strip is located on the front side of the external impermeable layer of the diaper immediately beneath a waist band region of the diaper.

In some exemplary embodiments, the enclosed accessory is one or more wet wipes.

In some exemplary embodiments, the accessory compartment strip includes decorative images.

In some exemplary embodiments, the pull tab is an adhesive strip that peels off.

### DETAILED DESCRIPTION

The present disclosure relates to a diaper having an enclosed accessory. The enclosed accessory is accessible through a pull tab that releases the enclosed accessory when the tab is pulled. During the removal of a diaper, the enclosed accessory is readily available for use; this mitigates the inconvenience of changing a diaper by combining both a diaper and an accessory used when changing a diaper in one efficient product.

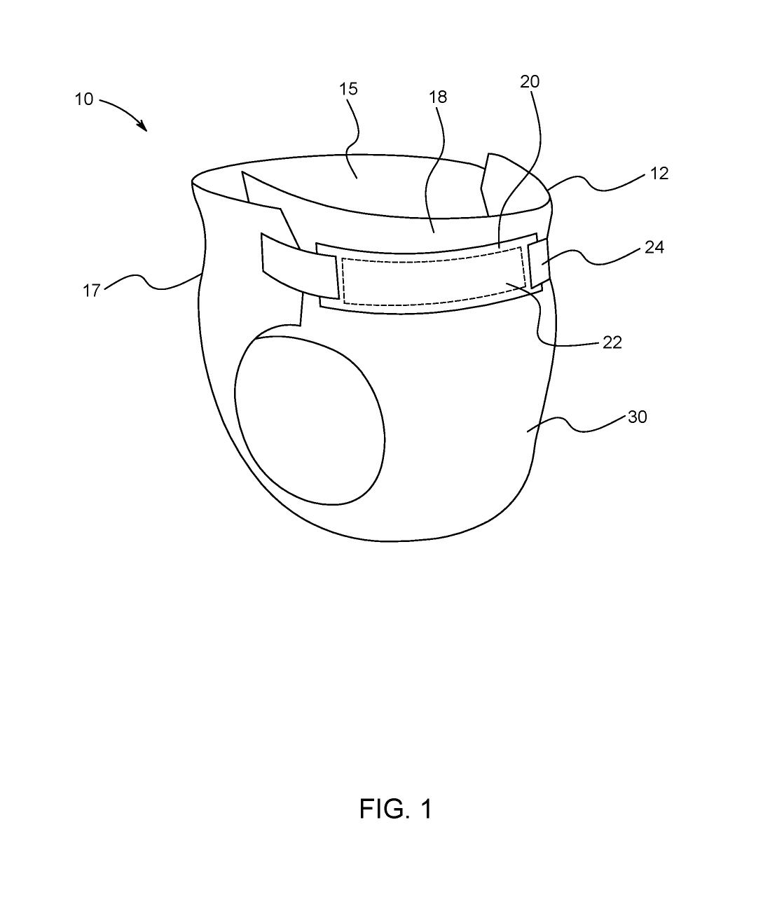

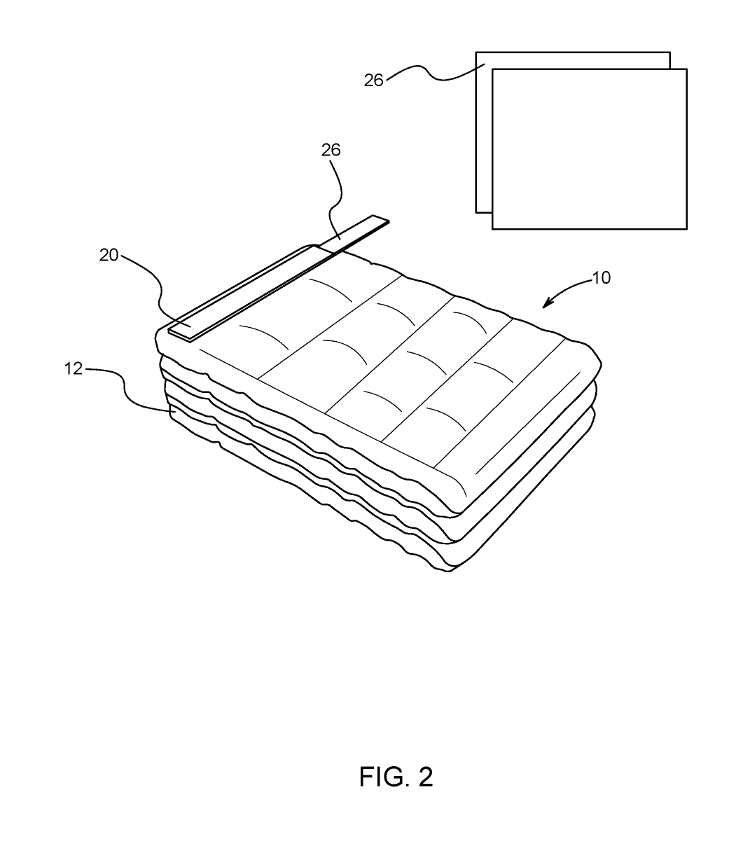

As illustrated in FIGS. 1-3, an exemplary diaper with an enclosed accessory 10 includes a diaper 12 having an internal absorbent layer 15 and an external impermeable layer 17. The external impermeable layer includes an accessory compartment strip 20 attached thereto. The accessory compartment strip includes an external pull tab layer 24 and an internal moisture retaining enclosure 22 for retaining an enclosed accessory 26.

The diaper 12 can be any suitable type of diaper. In a preferred embodiment, the diaper is a disposable diaper. The internal absorbent layer 15 is made of an absorbent material, such as, for example, polypropylene. The internal layer may also include wood pulp, absorbent polymers, and sodium polyacrylate, which can absorb thirty (30) times its weight in urine, thereby keeping the wearer of the diaper dry The external impermeable layer 17 is made of an impermeable material, such as, for example, a polyethylene film.

The accessory compartment strip 20 is located on a front side 30 of the external impermeable layer 17 of the diaper. In a preferred embodiment, the accessory compartment strip is located on the front side 30 of the external impermeable layer 17 of the diaper immediately below a waist band region 18 of the diaper. In some exemplary embodiments, the accessory compartment strip includes decorative images on the topmost external layer, such as, for example, cartoon characters.

The pull tab external layer 24 is an adhesive strip that peels off. The adhesive material can be any suitable material, such as, for example, acrylic.

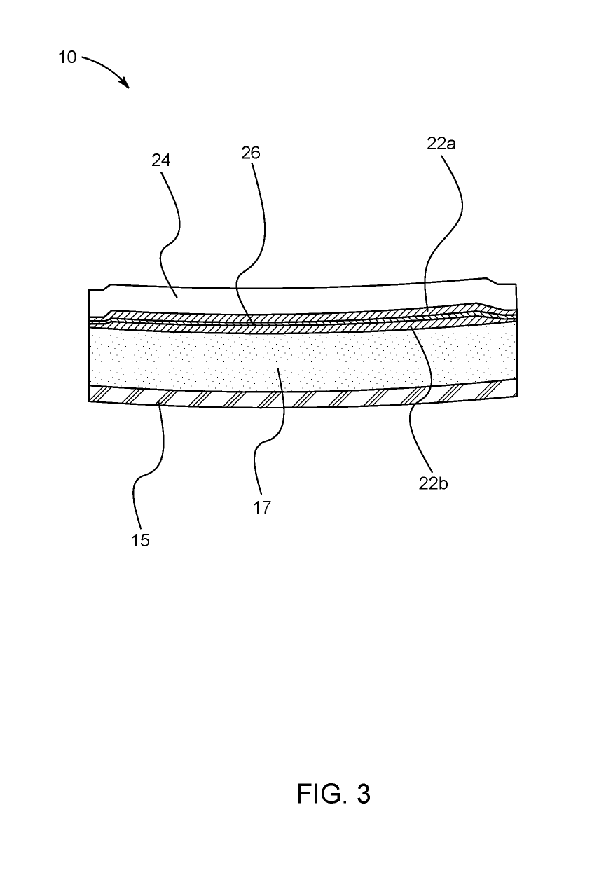

As illustrated in FIG. 3, the internal moisture retaining enclosure 22 includes a top release layer 22a and a bottom layer 22b and may be made of any suitable material that retains moisture, such as, for example, plastic. The top release layer 22a attaches directly to pull tab external layer 24. The top release layer 22a and the pull tab external layer 24 are attachably connected such that the top release layer is removed when the pull tab external layer is removed. The bottom layer 22b attaches directly to external impermeable layer 17. The internal moisture retaining enclosure 22 retains an accessory inside the enclosure (not shown in FIG. 3).

The accessory can be any suitable accessory. In an exemplary embodiment, the enclosed accessory 26 is a wet wipe (i.e., at least one). In a preferred embodiment, the enclosed accessory is a plurality of plurality of wet wipes. Wet wipes are used for cleaning the area covered by the diaper when the diaper is removed.

Although some of the exemplary embodiments described herein are tailored to a wet wipe as the enclosed accessory, the present disclosed diaper is not limited to such enclosed accessory, and can be used with any type of accessory including, ointments, creams, powder, etc.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.)

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

Those skilled in the art will appreciate from the foregoing description that the broad techniques of the embodiments of the present invention may be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

What is claimed is:

1. A diaper with an enclosed accessory comprising:
a diaper having an internal absorbent layer and an external impermeable layer, the external impermeable layer having an accessory compartment strip attached thereto;
the accessory compartment strip including a pull tab external layer attached to an internal moisture retaining enclosure which retains an accessory, the moisture retaining enclosure having a bottom layer attached directly to the external impermeable layer and a top release layer attached directly to the pull tab layer, wherein the top release layer releases the enclosed accessory when the pull tab is removed.

2. The diaper with an enclosed accessory of claim 1, wherein the accessory compartment strip is located on a front side of the external impermeable layer.

3. The diaper with an enclosed accessory of claim 2, wherein the accessory compartment strip is located on the front side of the external layer immediately beneath a waist band region of the diaper.

4. The diaper with an enclosed accessory of claim 1, wherein the enclosed accessory is one or more wet wipes.

5. The diaper with an enclosed accessory of claim 1, wherein the accessory compartment strip includes decorative images.

6. The diaper with an enclosed accessory of claim 1, wherein the pull tab is an adhesive strip that peels off.
